(19) United States
(12) Patent Application Publication
(10) Pub. No.: US 2020/0088713 A1
(43) Pub. Date: Mar. 19, 2020

(54) METHOD OF FORMING A NANOPORE AND RESULTING STRUCTURE

(71) Applicant: Applied Materials, Inc., Santa Clara, CA (US)

(72) Inventors: William J. DURAND, Oakland, CA (US); Joseph R. JOHNSON, Redwood City, CA (US); Roger QUON, Rhinebeck, NY (US)

(21) Appl. No.: 16/517,121

(22) Filed: Jul. 19, 2019

Related U.S. Application Data
(60) Provisional application No. 62/731,665, filed on Sep. 14, 2018.

(51) Int. Cl. G01N 33/487

(52) U.S. Cl. CPC G01N 33/48721; B82B 3/0019

(57) ABSTRACT

Methods are provided for manufacturing well-controlled, solid-state nanopores in close proximity and arrays thereof. In one embodiment, a plurality of wells and one or more channels are formed in a substrate. Each of the wells is adjacent a channel. A portion of a sidewall of each well is exposed. The portion of exposed sidewall is nearest to the adjacent channel. The portion of the exposed sidewall of each well is laterally etched towards the adjacent channel. A nanopore is formed connecting the wells to an adjacent channel.

### CROSS-REFERENCE TO RELATED APPLICATIONS

This application claims benefit of U.S. provisional patent application Ser. No. 62/731,665, filed Sep. 14, 2018, which is herein incorporated by reference.

### BACKGROUND

### Field

Aspects disclosed herein relate to methods of manufacturing well-controlled, solid-state nanopores and arrays of well-controlled, solid-state nanopores in a substrate.

### Description of the Related Art

Nanopores are widely used for applications such as deoxyribonucleic acid (DNA) and ribonucleic acid (RNA) sequencing. In one example, nanopore sequencing is performed using an electrical detection method, which generally includes transporting an unknown sample through a nanopore, which sample is immersed in a conducting fluid, and applying electric potential across the nanopore. Electric current resulting from the conduction of ions through the nanopore is measured. The magnitude of the electric current density across a nanopore surface depends on the nanopore dimensions and the composition of the sample, such as DNA or RNA, which is occupying the nanopore at the time. Different nucleotides cause characteristic changes in electric current density across nanopore surfaces. These electric current changes are measured and used to sequence the DNA or RNA sample.

Various methods have been used for biological and macromolecule sequencing. Sequencing by synthesis, or second generation sequencing, is used to identify which bases have attached to a single strand of DNA. Third generation sequencing, which generally includes threading an entire DNA strand through a single pore, is used to directly read the DNA. Some sequencing methods require the DNA or RNA sample to be cut up and then reassembled. Additionally, some sequencing methods use biological membranes and biological pores, which have shelf lives and must be kept cold prior to use.

Solid-state nanopores, which are nanometer-sized pores formed on a free-standing membrane such as a silicon containing material, have recently been used for sequencing. Current solid-state nanopore fabrication methods, such as using a tunneling electron microscope, focused ion beam, or electron beam, however, cannot easily and cheaply achieve the size and position control requirements necessary for manufacturing arrays of nanopores. Additionally, current nanopore fabrication methods are time consuming, and can be difficult to fabricate nanopores in close proximity to other nanopores.

Therefore, there is a need in the art for improved methods of manufacturing well-controlled, solid-state nanopores disposed in close proximity to one another.

### SUMMARY

In one aspect, a method for forming a plurality of nanopores comprises depositing a first layer on a substrate and forming a plurality of wells and one or more channels in the first layer and the substrate. Each of the plurality of wells is adjacent to a channel. The method further comprises laterally etching a portion of an exposed sidewall to connect the plurality of wells to the adjacent channel and forming nanopores connecting each of the plurality of wells to the adjacent channel.

In another aspect, a method for forming a plurality of nanopores comprises depositing a first layer on a substrate and forming a first well, a second well, and a channel in the first layer and the substrate. The channel is disposed adjacent to the first well and the second well. The method further comprises exposing a first portion of a sidewall in the first well and a second portion of a sidewall in the second well. The first portion of the exposed sidewall in the first well and the second portion of the exposed sidewall in the second well are adjacent the channel. A first tunnel is formed under the first layer extending from the first well and the channel. A second tunnel is formed under the first layer extending from the second well and the channel. A first nanopore connecting the first tunnel to the channel is formed and a second nanopore connecting the second tunnel to the channel is formed.

In yet another aspect, a device comprises a first well disposed within a substrate, a second well disposed within the substrate, and a channel disposed within the substrate adjacent to the first well and the second well. The substrate further comprises a first nanopore coupled to the first well and the channel and a second nanopore coupled to the second well and the channel. The second nanopore is disposed less than 1 μm from the first nanopore.

### BRIEF DESCRIPTION OF THE DRAWINGS

So that the manner in which the above recited features of the present disclosure can be understood in detail, a more particular description of the disclosure, briefly summarized above, may be had by reference to aspects, some of which are illustrated in the appended drawings. It is to be noted, however, that the appended drawings illustrate only exemplary aspects and are therefore not to be considered limiting of its scope, and may admit to other equally effective aspects.

FIG. 1 is a process flow of a method for forming a plurality of nanopores according to the present disclosure.

FIGS. 2A-2N depict top views and cross-sectional views of a chip in which a plurality of nanopores are formed according to a method disclosed herein.

FIGS. 3A-3F illustrate various embodiments of chips having various nanopore designs or layouts, according to various embodiments.

To facilitate understanding, identical reference numerals have been used, where possible, to designate identical elements that are common to the figures. It is contemplated that elements and features of one aspect may be beneficially incorporated in other aspects without further recitation.

### DETAILED DESCRIPTION

Methods are provided for manufacturing well-controlled, solid-state nanopores in close proximity and arrays thereof. In one embodiment, a plurality of wells and one or more channels are formed in a substrate. Each of the wells is adjacent a channel. A portion of a sidewall of each well is exposed, the portion of exposed sidewall being nearest to the adjacent channel. The portion of the exposed sidewall of each well is laterally etched towards the adjacent channel. A nanopore is then formed connecting each well to an adjacent channel. Each nanopore can be spaced a distance less than 1 μm from adjacent nanopores.

Methods disclosed herein refer to formation of solid-state nanopores on a semiconductor chip as an example. It is also contemplated that the disclosed methods are useful to form other microfluidic devices and pore-like structures on various materials, including solid-state and biological materials. Methods disclosed herein also refer to formation of pyramid-shaped tunnels as an example; however, other etched features and any combinations thereof are also contemplated. For illustrative purposes, a silicon substrate is described; however, any suitable substrate materials and dielectric materials, such as glass, are also contemplated.

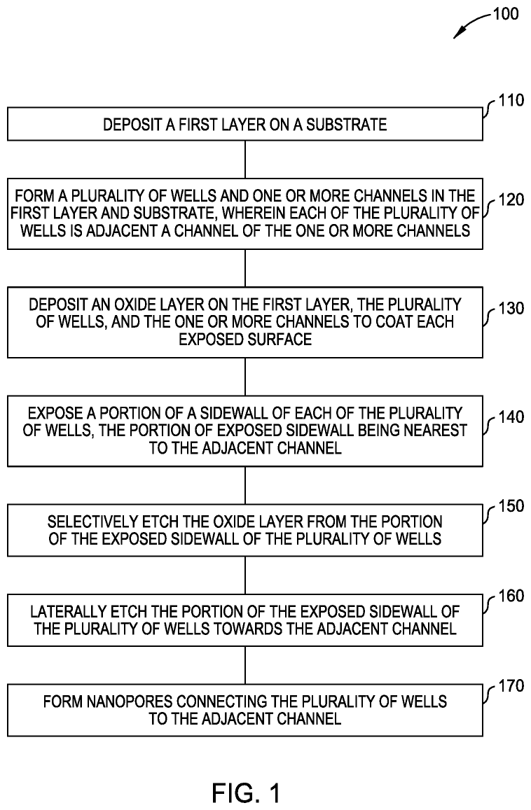

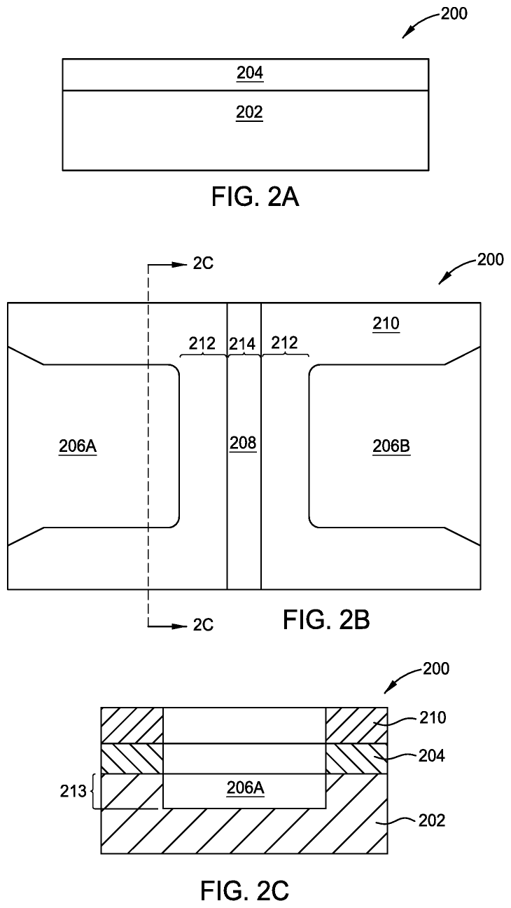

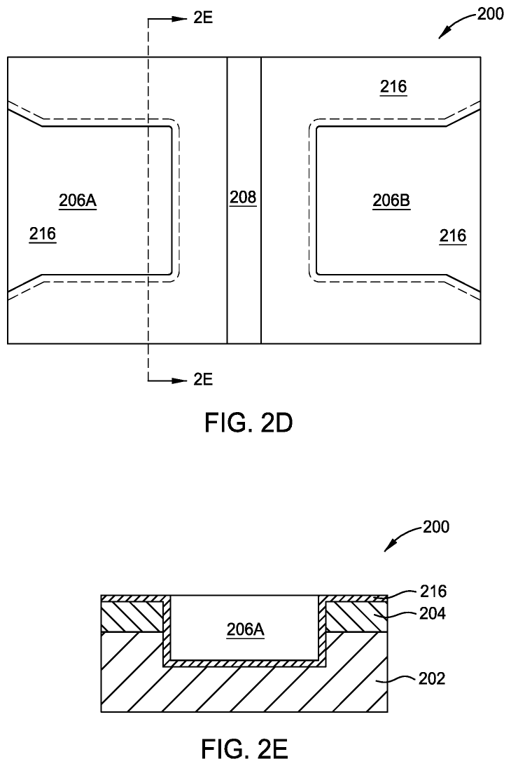

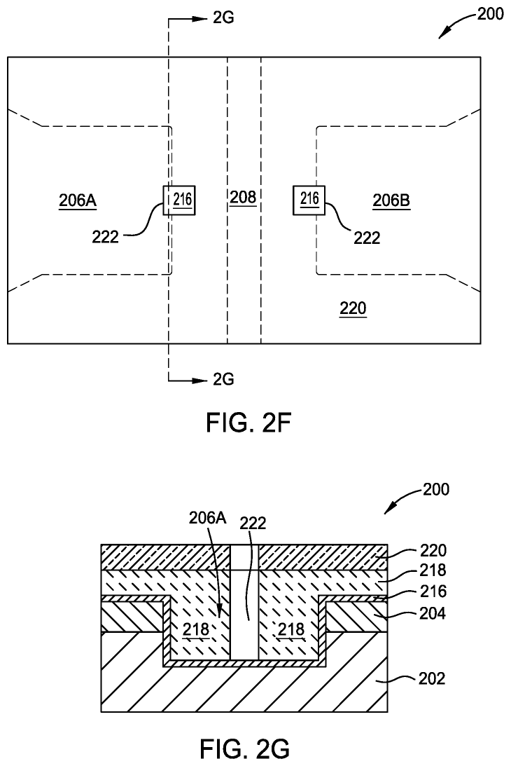

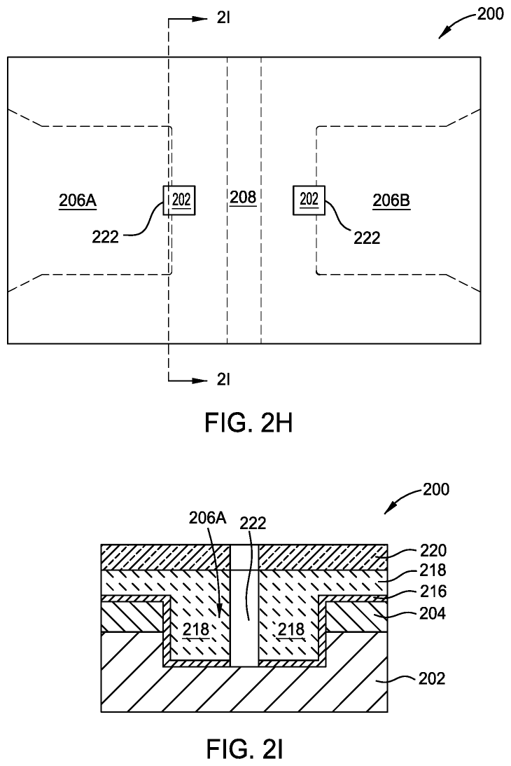

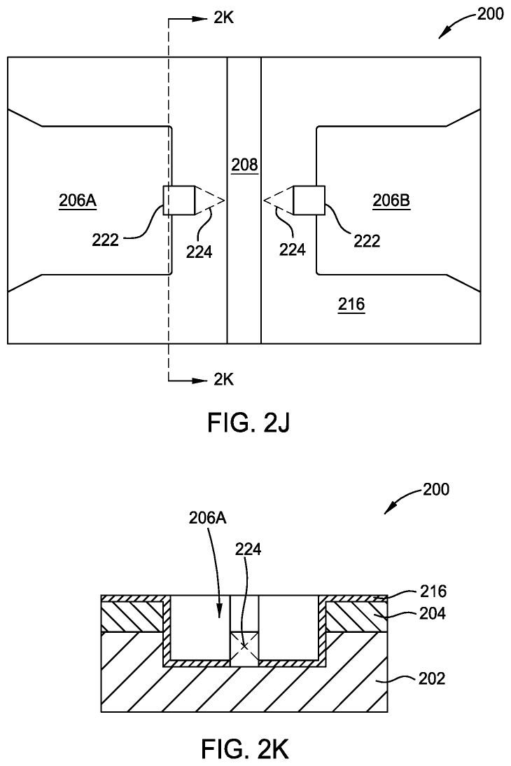

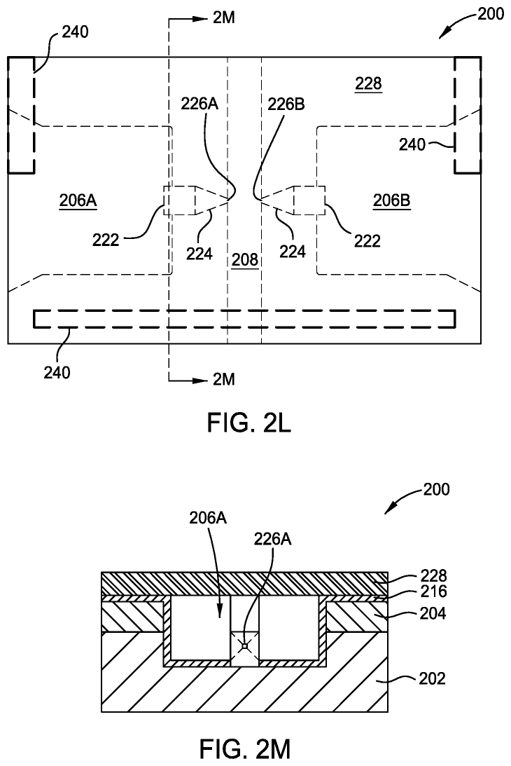

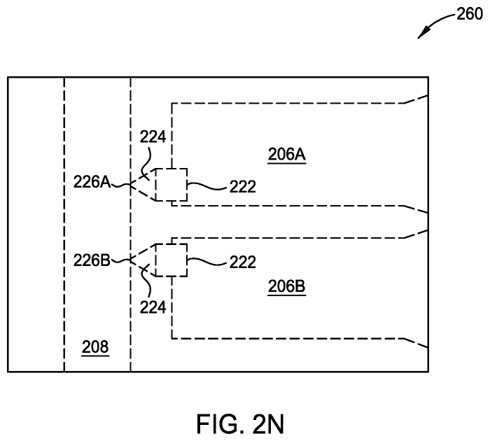

FIG. 1 is a process flow of a method 100 for forming a plurality of nanopores according to the present disclosure. FIGS. 2A-2N depict top views and cross-sectional views of a chip 200 in which a plurality of nanopores are formed according to a method disclosed herein, such as at various stages of the method 100. While FIGS. 2A-2N are shown in a particular sequence, it is also contemplated that the various stages of method 100 depicted in FIGS. 2A-2N can be performed in any suitable order. To facilitate a clearer understanding of the method 100, the method 100 of FIG. 1 will be described and demonstrated using the various views of the chip 200 in FIGS. 2A-2N. While the method 100 is described using FIGS. 2A-2N, other operations not shown in FIGS. 2A-2N may be included.

Prior to method 100, a substrate 202 is provided. The substrate 202 is generally any suitable semiconductor substrate, such as a doped or undoped silicon (Si) substrate. The substrate 202 may have thickness between 200 μm to 2000 μm. In one embodiment, the substrate 202 is Si having a crystal structure including a <100> plane. In operation 110, a first layer 204 is deposited on the substrate 202, as shown in the cross-sectional view of FIG. 2A. The first layer 204 may function as a hard mask. In at least one implementation, the first layer 204 is a potassium hydroxide (KOH) resistant etch barrier, such as silicon nitride (SiN). The first layer 204 may have a thickness between about 1 nm to about 100 nm. In one embodiment, the first layer 204 has a thickness of about 50 nm. The first layer 204 is generally deposited by any suitable deposition methods, including but not limited to, atomic layer deposition (ALD), physical vapor deposition (PVD), or chemical vapor deposition (CVD).

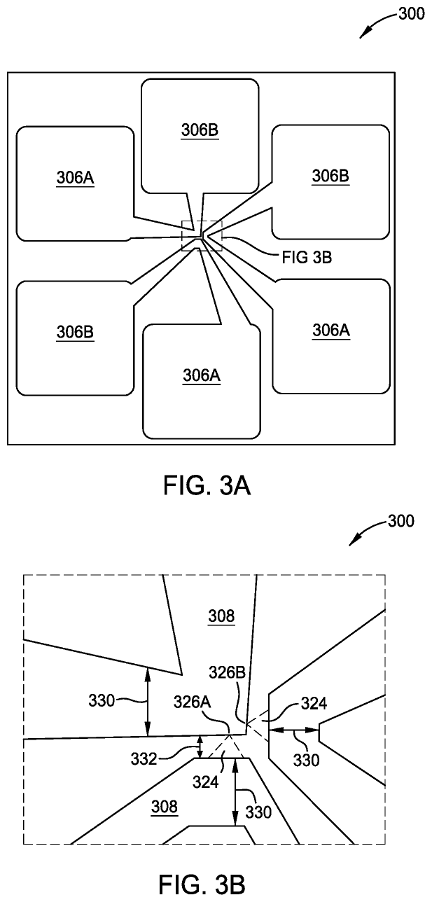

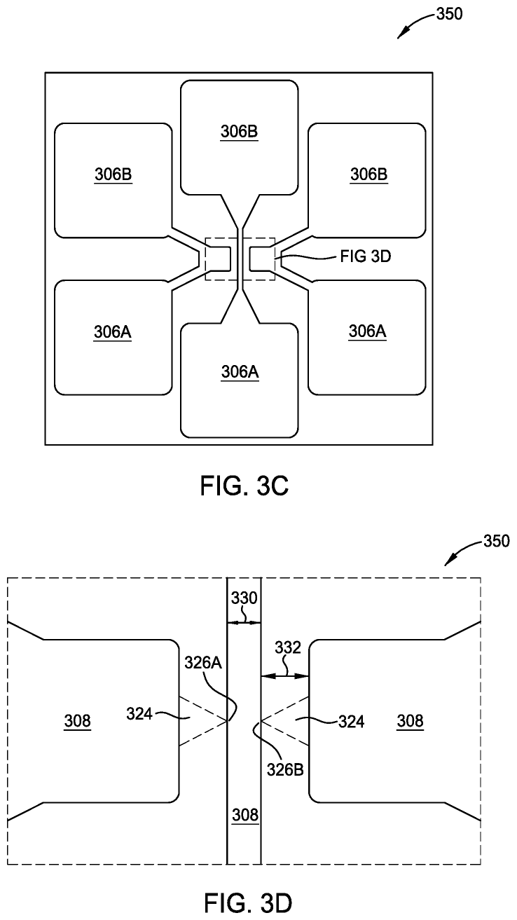

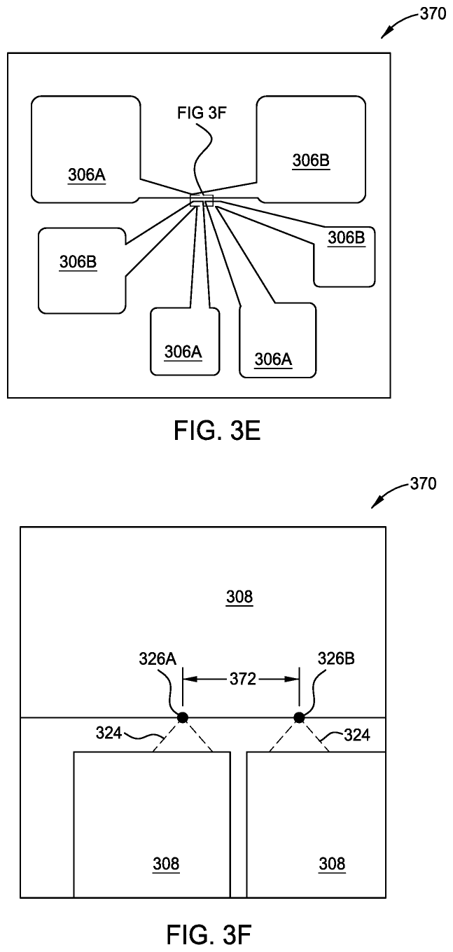

In operation 120, a plurality of wells 206A-206B and one or more channels 208 are formed, as shown in FIGS. 2B-2C. FIG. 2B is a top view of the chip 200 while FIG. 2C is cross-sectional through the line labeled 2C in FIG. 2B. Each of the plurality of wells 206A-20B are disposed adjacent a channel 208 of the one or more channels. In at least one implementation, an even number of wells are formed on the chip 200. While only two wells 206A-206B and one channel 208 are shown, any number of wells and channels may be utilized, as shown and described in FIGS. 3A-3B below. Forming at least two wells 206A-206B, or an even number of wells, allows the wells (and later, the nanopores coupled to the wells) to be utilized in pairs.

To form the wells 206A-206B and channel 208 in operation 120, a first photoresist layer 210 is deposited on the first layer 204. A patterning process is then performed to form the wells 206A-206B and channel 208. Generally, the patterning process includes lithographing or patterning the first photoresist layer 210 and etching, for example by reactive ion etching (RIE), the first layer 204 and the substrate 202. The etching may be a directional etch. The first photoresist layer 210 is then removed.

The wells 206A-206B and channel 208 may be etched to a depth 213 between 10 nm to 2 μm. In one embodiment, the wells 206A-206B and channel 208 are etched to have a depth 213 of about 250 nm. The wells 206A-206B may be spaced a distance 212 of between 20 nm to 500 nm away from the channel 208. The channel 208 may have a width 214 of about 1 nm to 200 nm. In one embodiment, the channel 208 may have a width 214 of less than 100 nm. Thus, the first well 206A may be spaced a distance of less than 1000 nm from the second well 206B.

In operation 130, a second layer 216, such as a material which exhibits a suitable degree of etch selectivity relative to the first layer 204, for example, an oxide layer, is deposited or grown on the first layer 204, the plurality of wells 206A-206B, and the channel 208 to coat each exposed surface of the chip 200, as shown in FIGS. 2D-2E. FIG. 2D is a top view of the chip 200 while FIG. 2E is cross-sectional through the line labeled 2E in FIG. 2D. The second layer 216 is deposited in a conformal layer over each exposed surface of the chip 200. The second layer 216 may have a thickness between 1 nm to 100 nm. In one aspect, the second layer 216 has a thickness between 5 nm to 10 nm. In one embodiment, the first layer 204 is oxidized, for example by exposing the first layer 204 to oxygen or water (H₂O), to form the second layer 216. In another embodiment, the second layer 216 is deposited using ALD. In yet another embodiment, the second layer 216 is formed by depositing a metal or semiconductor layer, for example, by ALD, CVD, or PVD, and then oxidizing the metal or semiconductor layer to form the second layer 216.

The second layer 216 may be a KOH etch-resistant layer. In at least one implementation, the second layer 216 comprises SiN. The second layer 216 may be base resistant. The second layer 216 generally comprises any suitable dielectric material with an etch rate that is low relative to SiO₂. Examples of suitable materials for the second layer 216 further include, but are not limited to, Al₂O₃, Y₂O₃, and TiO₂. The etch rate of the second layer 216 compared to the etch rate of SiN is generally greater than about 10:1, for example about 100:1, for example about 1,000:1.

In operation 140, a portion of the sidewall 222 of each of the wells 206A-206B is exposed, as shown in FIGS. 2F-2G. FIG. 2F is a top view of the chip 200 while FIG. 2G is cross-sectional through the line labeled 2G in FIG. 2F. The portion of exposed sidewall 222 is adjacent the channel 208, and is part of the substrate 202. In one embodiment, one or more portions of a sidewall of the channel 208 are exposed. In such an embodiment, a first portion of the sidewall of the channel 208 adjacent to the first well 206A is exposed, and a second portion of the sidewall of the channel 208 adjacent the second well 206B is exposed. The first portion of the sidewall and the second portion of the sidewall of the channel 208 may be disposed directly across from one another. The first portion of the sidewall and the second portion of the sidewall of the channel 208 may be disposed adjacent to one another.

To expose the portion of the sidewall 222, a second patterning process is performed. In the second patterning process, a planarization layer 218 is deposited to provide a planar surface for improved photolithography processes. A second photoresist layer 220 is then deposited on the planarization layer 218. A mask may be aligned with the portions of the sidewall 222 to be exposed. The second patterning process includes lithographing or patterning the second photoresist layer 220 and the planarization layer 218. The second patterning process further includes etching, for example by RIE or by a wet etching process, the second photoresist layer 220 and the planarization layer 218 to expose the portion of the sidewall 222 of the wells 206A-206B.

In operation 150, the second layer 216 is selectively etched from the portions of exposed sidewall 222 of the wells 206A-206B, as shown in FIGS. 2H-2I. FIG. 2H is a top view of the chip 200 while FIG. 2I is cross-sectional through the line labeled 21 in FIG. 2H. In an embodiment where portions of the sidewall of the channel 208 are exposed in operation 140, the second layer 216 is selectively etched from the portions of exposed sidewall of the channel 208.

To remove the second layer 216 from the portions of exposed sidewall 222, a wet etchant is utilized in one embodiment. For example, a fluoride based etchant, such as dilute hydrofluoric acid (DHF), may be used since oxide is selective to fluoride etches. In another embodiment, an isotropic dry etchant is utilized to remove the second layer 216 from the portions of the exposed sidewall 222. For example, the dry etchant may include a fluorine containing vapor or plasma. In one example, the fluorine containing vapor or plasma includes fluorine ions and/or fluorine radicals. The selective etch may remove the second layer 216 while leaving the first layer 204 intact. The second layer 216 may be selectively removed from the portions of exposed sidewall 222 while retaining the second layer 216 on the side surfaces of the wells 206A-206B, as shown in FIG. 2I. The second photoresist layer 220 and the planarization layer 218 may then be removed. By removing the second photoresist layer 220 and the planarization layer 218, the chip 200 has a base resistant second layer 216 on the non-exposed portions of the sidewalls of the wells 206A-206B and exposed silicon crystal surface on the portions of exposed sidewall 222.

In operation 160, the portions of exposed sidewall 222 are laterally etched towards the channel 208. The lateral etchant may comprise a basic liquid chemistry, for example a KOH dip or by exposure to tetramethylammonium hydroxide (TMAH), as shown in FIGS. 2J and 2K. FIG. 2J is a top view of the chip 200 while FIG. 2K is cross-sectional through the line labeled 2K in FIG. 2J. In one embodiment, the lateral etchant comprises an anisotropic etch. In another embodiment, the lateral etchant comprises an isotropic etch. In an embodiment where portions of the sidewall of the channel 208 are exposed in operation 140, the portions of exposed sidewall of the channel 208 are laterally etched towards the wells 206A-206B.

The lateral etch comprises etching the substrate 202 in a manner parallel to a planar upper surface of the substrate 202. The lateral etch may be an anisotropic etch. Laterally etching the portions of exposed sidewall 222 towards the channel 208 forms tunnels 224 or paths through the substrate 202 under the first layer 204. The tunnels 224 are pyramid or frustum-shaped, and are parallel to a planar upper surface of the first layer 204. The size of the tunnels 224 may vary depending on the size of the portions of exposed sidewall 222. The tunnels 224 may be etched until only a thin film membrane of the second layer 216 remains between the tunnels 224 and the channel 208.

The lateral etch may be performed for a predetermined amount of time to etch the substrate 202 along the crystal facets or lattice of the crystal structure. The predetermined period of time is generally determined to reduce or eliminate lateral etch relative to the mask opening. In general, the <100> plane of the Si substrate 202 will etch at a rate that corresponds to the temperature of the solution and the concentration of KOH in H₂O. For most scenarios, KOH will etch the <100> plane of Si at a rate of between about 0.4 nm/s and about 20 nm/s. The rate can be accelerated or retarded by cooling or heating the solution. The portions of the exposed sidewalls 222 may be exposed to the etchant for 0.5 to 5 minutes at a temperature of 0 to 100 degrees Celsius. In one embodiment, a 30% weight of aqueous KOH solution is heated to about 40 degrees, and is applied for about 1 minute.

In operation 170, a plurality of nanopores 226A-226B is formed to connect the tunnels 224 to the channel 208, as shown in FIGS. 2L-2N. FIG. 2L is a top view of the chip 200 while FIG. 2M is cross-sectional through the line labeled 2M in FIG. 2L. FIG. 2N illustrates an embodiment of a chip 260 having the wells 206A-206B being disposed on the same side of the channel 208 with the nanopores 226A-226B being substantially parallel or co-axially aligned. The chip 260 of FIG. 2N may be formed according to the method 100 as described with respect to FIGS. 2A-2M.

The nanopores 226A-226B may be formed by applying voltage to induce dielectric breakdown of the thin film membrane of the second layer 216 remaining between the tunnels 224 and the channel 208, resulting in forming well-controlled, localized, and robust nanopores. The nanopores 226A-226B are formed at the tip of the pyramid or frustum-shaped tunnels 224. One or more electrodes 240 may optionally be formed on the chip 200 in order to apply the voltage. The one or more electrodes 240 may be disposed on the second layer 216, within the wells 206A-206B, and within the channel 208. The one or more electrodes 240 may then be removed following the formation of the nanopores 226A-226B. In another embodiment, the chip 200 comprises electrodes configured to apply the voltage. A glass slide 228 may be deposited on and bonded to the second layer 216.

The applied voltage generally removes at least a portion of the second layer 216 to form the nanopores 226A-226B, for example, by degrading a portion of the second layer 216. The applied voltage generally includes typical voltages above the breakdown voltage of the second layer 216. For example, the breakdown voltage of silicon oxide is generally between about 2 megavolts (MV)/cm and about 6 MV/cm, or between about 200-600 millivolts (mV)/nm of material. In one aspect, the applied voltage is slightly below the breakdown voltage of the second layer 216 and the current is applied for longer to slowly break down the remaining membrane. In another aspect, the applied voltage is above the breakdown voltage of the substrate material such that the nanopores 226A-226B are blasted therethrough. If the nanopores 226A-226B are formed having a larger size than desired, an oxidation process may be performed to reduce the size of the nanopores 226A-226B. For example, the tip of the pyramid or frustum-shaped tunnels 224 may be oxidized to reduce the size of the nanopores 226A-226B. In one embodiment, the second layer 216 is not deposited on or is removed from a portion of the channel 208 disposed between the tunnels 224. In such an embodiment, the nanopores 226A-226B may be formed using the lateral etch of operation 160, and a voltage need not be applied to form the nanopores 226A-226B.

Forming at least two wells 206A-206B, and subsequently at least two nanopores 226A-226B, allows the nanopores 226A-226B coupled to the wells 206A-206B to be utilized in pairs, or as dual pores, to sequence macromolecules, such as proteins, and/or biological polymers, such as DNA. For example, the chip 200 may be filled with an electrolyte or conductive fluid comprising biological polymers and/or macromolecules. Single strands of DNA or macromolecules may be passed through the nanopore 226A coupled to the first well 206A through the nanopore 226B coupled to the second well 206B to determine properties of or materials attached to the biological polymers and/or macromolecules. The electric properties include an electric signal, which may change based on the size and/or shape of the DNA base pair. The nanopore 226A coupled to the first well 206A may control the collection rate at which biological polymers and/or macromolecules can be attracted to the nanopore 226A, and the nanopore 226B coupled to the second well 206B may control the speed or rate at which biological polymers and/or macromolecules is passed through the nanopore 226B, or vice versa. In another embodiment, both nanopores 226A, 226B influence the speed at which the biological polymers and/or macromolecules is passed therethrough via application of electric fields having different magnitudes. Thus, utilizing dual nanopores allows the dual nanopores to be in fluid communication with one another, resulting in improved signal-to-noise ratios and a higher capturing rate of the biological polymers and/or macromolecules while still maintaining control.

Because the nanopores 226A-226B have been formed according to methods disclosed herein, the size and position of the nanopores 226A-226B are well controlled. A well-controlled size of the nanopores 226A-226B is generally a diameter suitable for sequencing a sample of a certain size. In one aspect, the size of the nanopores 226A-226B is about 100 nm or less. In one aspect, the nanopores 226A-226B are between about 5 nm by 5 nm and about 50 nm by 50 nm. In one embodiment, the nanopores 226A-226B have a diameter between about 5 nm and 50 nm. In one embodiment, the nanopores 226A-226B are about 20 nm by 20 nm. In another aspect, the size of the nanopores 226A-226B is between about 1.5 nm and about 1.8 nm, such as about 1.6 nm, which is roughly the size of a single strand of DNA. In another aspect, the size of the nanopores 226A-226B is between about 2 nm and about 3 nm, such as about 2.8 nm, which is roughly the size of double-stranded DNA. A well-controlled position of the nanopores 226A-226B is generally any position on the substrate which is suitable for configuration of one or more nanopores. In one embodiment, the nanopores 226A-226B are spaced less than 1 μm away from each other, for example less than 100 nm away from each other.

In one aspect, the chip 200 includes an array of nanopores 226, as shown in FIGS. 3A-3F. Methods disclosed herein are generally used to control the position of each of the plurality of nanopores 226 such that a nanopore array of desired configuration for sequencing or other processes is formed. Method 100 is not limited to the above described operations, and may include one or more various other operations.

FIGS. 3A-3F illustrate various embodiments of chips 300, 350, respectively, having a plurality of nanopores in various designs or layouts, according to various embodiments. The chips 300 and 350 may be the chip 200 of FIGS. 2A-2N. Additionally, the channels 308, the tunnels 324, the wells 306A-306B, and the nanopores 326A-326B of FIGS. 3A-3F may be the channels 208, the tunnels 224, the wells 206A-206B, and the nanopores 226A-226B of FIGS. 2A-2N, respectively.

In FIGS. 3A-3B, a chip 300 comprises an array of well pairs in a right-angle design. The chip 300 illustrates three pairs of wells 306A-306B coupled to nanopores, with each well 306A-306B being coupled to a channel 308 by a tunnel 324. FIG. 3B illustrates a close up of the nanopores 326A-326B in the center of the chip 300 of FIG. 3A. As shown in FIG. 3B, the nanopores 326A and 326B are disposed at substantially right angles to one another. In one embodiment, each of the three pairs of wells 306A-306B has a distinct function for sequencing biological polymers and/or macromolecules, such as providing different fluid and electrical access to the biological polymers and/or macromolecules. For example, after the nanopores 326A-326B have been formed on the chip 300, a sample-containing solution is generally deposited in a first set of wells 306A-306B and a sample-free solution is deposited over a second set of wells 306A-306B.

Each channel 308 of the chip 300 may narrow as the channel 308 extends towards the center of the chip 300. The channels 308 may have a width 330 of about 1 μm to 20 μm. In one embodiment, the channels 308 have a width 330 of about 10 μm. The tunnels 324 may have a length 332 extending from one channel 308 to another channel 308 of about 0.1 μm to 0.5 μm. In one embodiment, the tunnels 324 have a length 332 of about 0.25 μm. In another embodiment, the nanopores 326A-326B are spaced less than 1 μm away from each other, for example less than 100 nm away from each other. In FIGS. 3A-3B, the channels 308 have a width of up to 20 μm while still permitting the nanopores 336A-33B to be spaced less than 1 μm away each other.

Since the nanopores 326A-326B are disposed at substantially right angles to one another, the distance between the nanopores 326A and 326B does not depend on the width 330 of a channel 308, as the nanopores 326A-326B are not separated by the channel 308. Having wider channels 308 enables the tunnels 324 to be larger as well. Utilizing a chip 300 having closely spaced nanopores 326A-326B and larger tunnels 324 and channels 308 allows for a greater amount of fluid to pass through the channels 308 and tunnels 324, resulting in less electrical resistance being encountered when sequencing biological polymers and/or macromolecules. As such, higher flow rates and enhanced electrical properties may be achieved, and larger biological polymers and/or macromolecules may be sequenced.

In FIGS. 3C-3D, a chip 350 comprises an array of well pairs in a parallel or co-axially aligned design, according to one embodiment. The chip 350 illustrates three pairs of wells 306A-306B coupled to nanopores, with each well 306A-306B being coupled to a channel 308 by a tunnel 324. FIG. 3D illustrates a close up of the nanopores 326A-326B in the center of the chip 350 of FIG. 3C. As shown in FIG. 3D, the nanopores 326A and 326B are disposed substantially parallel or co-axially aligned with one another. In one embodiment, each of the three pairs of wells 306A-306B has a distinct function for sequencing biological polymers and/or macromolecules, such as providing different fluid and electrical access to the biological polymers and/or macromolecules. For example, after the nanopores 326A-326B have been formed on the chip 300, a sample-containing solution is generally deposited in a first set of wells 306A-306B and a sample-free solution is deposited over a second set of wells 306A-306B.

In FIGS. 3E-3F, a chip 370 comprises an array of well pairs in an in-plane or co-axially aligned design, according to another embodiment. The chip 370 illustrates three pairs of wells 306A-306B coupled to nanopores, with each well 306A-306B being coupled to a channel 308 by a tunnel 324. FIG. 3F illustrates a close up of the nanopores 326A-326B in the center of the chip 370 of FIG. 3E. As shown in FIG. 3F, the nanopores 326A and 326B are disposed substantially in-plane or co-axially aligned with one another. The nanopores 326A and 326B are disposed adjacent or substantially parallel to one another. The nanopores 326A and 326B may be spaced a distance 372 from one another. Similar to the chip 300, the distance 372 the nanopores 326A-326B are spaced from one another does not depend on the width of a channel 308, as the nanopores 326A-326B are not separated by the channel 308. Thus, higher flow rates and enhanced electrical properties may be achieved, and larger biological polymers and/or macromolecules may be sequenced.

In one embodiment, each of the three pairs of wells 306A-306B has a distinct function for sequencing biological polymers and/or macromolecules, such as providing different fluid and electrical access to the biological polymers and/or macromolecules. For example, after the nanopores 326A-326B have been formed on the chip 300, a sample-containing solution is generally deposited in a first set of wells 306A-306B and a sample-free solution is deposited over a second set of wells 306A-306B.

The embodiments of FIGS. 3A-3F are but three examples of chips having dual nanopore designs, and are not limited to the above embodiments. Any suitable dual nanopore layouts or designs are also contemplated.

Benefits of the present disclosure include the ability to quickly form well-controlled nanopores and nanopore arrays having nanopore pairs formed in close proximity. Disclosed methods generally provide nanopores that are well-controlled in size and in position through a thin film membrane. Methods of manufacturing nanopores of well-controlled size provide improved signal-to-noise ratios and higher biological polymers and/or macromolecules capturing rates while maintaining a high level of control. Single strands of biological polymers and/or macromolecules are able to be captured at a higher collection rate and are able to be transmitted through the nanopores at increased speeds, which increases the change in electric current passing through the nanopore. Therefore, utilizing well-controller nanopore pairs provides for improved reading of the DNA sequence.

While the foregoing is directed to aspects of the present disclosure, other and further aspects of the disclosure may be devised without departing from the basic scope thereof, and the scope thereof is determined by the claims that follow.

What is claimed is:

1. A method for forming a plurality of nanopores, comprising:
depositing a first layer on a substrate;
forming a plurality of wells and one or more channels in the first layer and the substrate, each of the plurality of wells being adjacent a channel of the one or more channels;
laterally etching a portion of an exposed sidewall to connect the plurality of wells to the adjacent channel; and
forming nanopores connecting each of the plurality of wells to the adjacent channel.

2. The method of claim 1, further comprising depositing a second layer on the first layer, the plurality of wells, and the one or more channels to coat each exposed surface prior to exposing the portion of the sidewall of each of the plurality of wells.

3. The method of claim 2, further comprising selectively etching the second layer from the portion of the exposed sidewall prior to laterally etching the portion of the exposed sidewall.

4. The method of claim 3, wherein the second layer is an oxide comprising layer.

5. The method of claim 3, wherein selectively etching the second layer comprises a liquid acidic etch.

6. The method of claim 1, wherein the substrate comprises a crystal structure.

7. The method of claim 6, wherein laterally etching the portion of the exposed sidewall of the plurality of wells comprises a basic wet etch along the crystal structure of the substrate.

8. The method of claim 1, wherein forming the nanopores comprises applying a voltage.

9. A method for forming a plurality of nanopores, comprising:
depositing a first layer on a substrate;
forming a first well, a second well, and a channel in the first layer and the substrate, the channel being disposed adjacent to the first well and the second well;
forming a first tunnel under the first layer, the first tunnel extending between the first well and the channel;
forming a second tunnel under the first layer, the second tunnel extending between the second well and the channel; and
forming a first nanopore connecting the first tunnel to the channel and a second nanopore connecting the second tunnel to the channel.

10. The method of claim 9, wherein the first nanopore is disposed less than 1 μm from the second nanopore.

11. The method of claim 9, wherein the first nanopore is disposed substantially parallel to the second nanopore.

12. The method of claim 9, wherein the first nanopore is disposed at a substantially right angle to the second nanopore.

13. The method of claim 9, further comprising depositing a second layer on the first layer, the first well, the second well, and the channel to coat each exposed surface prior to forming the first tunnel and the second tunnel under the first layer.

14. The method of claim 13, further comprising selectively etching the second layer from a first portion of an exposed sidewall of the first well and a second portion of an exposed sidewall of the second well prior to forming the first tunnel and the second tunnel under the first layer.

15. The method of claim 9, wherein the first tunnel and the second tunnel are formed by a lateral etch.

16. The method of claim 15, wherein the lateral etch comprises a basic wet etch along a crystal structure of the substrate.

17. A device, comprising:
a first layer disposed on a substrate;
a first well disposed through the first layer within the substrate;
a second well disposed through the first layer within the substrate;
a channel disposed through the first layer within the substrate adjacent to the first well and the second well;
a first laterally etched nanopore coupled to the first well and the channel; and
a second laterally etched nanopore coupled to the second well and the channel, the second nanopore being disposed less than 1 μm from the first nanopore.

18. The substrate of claim 17, wherein the laterally etched first nanopore is coupled to the first well through a first pyramid shaped tunnel and the laterally etched second nanopore is coupled to the second well through a second pyramid shaped tunnel.

19. The substrate of claim 17, wherein the first well is disposed less than 1000 nm from the second well.

20. The substrate of claim 17, wherein the second nanopore is disposed less than 1000 nm from the first nanopore.
